(19) United States
(12) Patent Application Publication
(10) Pub. No.: US 2010/0157247 A1
(43) Pub. Date: Jun. 24, 2010

(54) OPHTHALMIC ILLUMINATION FILTER SELECTION MECHANISM

(76) Inventors: Simon Roderick Grover, Cambridge (GB); David John Stocks, Melbourn (GB)

Correspondence Address: Bausch & Lomb Incorporated, One Bausch & Lomb Place, Rochester, NY, 14604-2701, US

(21) Appl. No.: 12/339,322

(22) Filed: Dec. 19, 2008

(51) Int. Cl. A61B 3/10

(52) U.S. Cl. 351/213

(57) ABSTRACT

An ophthalmic illumination filter selection mechanism for use with an ophthalmic illumination system. Three filters are mounted in holders on a first shaft and rotatable into an optical light path determined by the ophthalmic illumination system. Each of three cams mounted on a second shaft is configured to move a holder on the first shaft to position the corresponding filter in the optical light path in a predefined sequence relative to the other filters as the second shaft is rotated in a forward direction. Each cam has a backlash that allows the second shaft to be rotated in a reverse direction for a predetermined radial distance to allow a last remaining filter to be removed from the optical light path. Each cam has a cam profile that ensures one filter remains in the optical light path until the next filter is driven into the optical light path when the second shaft is rotated in the forward direction. This mechanism occupies less space than conventional filter selection mechanisms.

### FIELD

The present disclosure relates to ophthalmic illumination systems and more particularly to an ophthalmic illumination filter selection mechanism.

### BACKGROUND

This section provides background information related to the present disclosure which is not necessarily prior art.

When ophthalmic surgery is performed, an ophthalmic illumination system is used to illuminate the interior of a patients eye so that the surgeon may view the surgical site. An ophthalmic illumination system typically includes a light source to which is coupled a length of optical fiber. Light travels through the fiber to the tip of an endo-illuminator, or probe, inserted into an incision in the eye.

Surgeons can obtain enhanced visualization of various eye features if a nominally white light source is filtered to provide various colored tints. A filtering device may be used to allow a surgeon to select one or more color tints during surgery. For example, a disk in which several color filters are circularly arranged may be rotated to place a filter that produces a selected tint in the path of the light source. Alternatively, a surgeon may select one of several color filters linearly arranged in an oblong slider by moving the slider linearly in front of the light source. Filter disks and sliders, however, tend to occupy considerable space when inserted into ophthalmic illumination systems.

### SUMMARY

This section provides a general summary of the disclosure, and is not a comprehensive disclosure of its full scope or all of its features.

In one configuration, the present disclosure is directed to an ophthalmic illumination filter selection mechanism for use with an ophthalmic illumination system. The mechanism includes a plurality of filters each mounted in a corresponding holder. Each holder is mounted on a first shaft and rotatable on the first shaft into an optical light path determined by the ophthalmic illumination system. A plurality of cams are mounted on a second shaft. Each cam is configured to move a corresponding holder on the first shaft to position the corresponding filter in the optical light path in a predefined sequence relative to the other filters as the second shaft is rotated in a forward direction. Each cam has a cam profile that ensures one of the filters remains in the optical light path until the next filter in the predefined sequence is driven into the optical light path. Each cam has a pin extending into a corresponding slot on the second shaft. The pin and corresponding slot are configured to allow the second shaft to be rotated relative to the cam for a predetermined radial distance to allow a last remaining filter to be removed from the optical light path when the second shaft is rotated in a reverse direction.

In another configuration, the disclosure is directed to an ophthalmic illumination filter selection mechanism for use with an ophthalmic illumination system. The mechanism includes a plurality of filters each mounted in a corresponding holder. Each holder is mounted on a first shaft and rotatable on the first shaft into a collimated portion of an optical light path determined by the ophthalmic illumination system. A plurality of cams are mounted on a second shaft. Each cam is drivable by the second shaft to move a corresponding holder on the first shaft to position the corresponding filter in the optical light path in a predefined sequence relative to the other filters as the second shaft is rotated in a forward direction. Each cam has a cam profile that ensures one of the filters remains in the optical light path until the next filter in the predefined sequence is driven into the optical light path. Each cam has a backlash configured to allow the second shaft to be rotated in a reverse direction for a radial distance to allow a last remaining filter to be removed from the optical light path. Additionally, the radial distance is predetermined to allow one of the filters to be rotated away from the optical light path without another filter being driven into the optical light path by rotation of the second shaft in a reverse direction.

In yet another configuration, the disclosure is directed to an ophthalmic illumination system having a light source and a light probe that receives light from the light source via an optical light path. The system includes a color filter selection mechanism having three filters. Each filter is mounted in a corresponding holder rotatable on a first shaft into a collimated portion of the optical light path. Three cams are mounted on a second shaft of the filter selection mechanism, each cam corresponding to one of the holders and drivable by the second shaft to move the corresponding holder on the first shaft to position the corresponding filter in the optical light path. The positioning is performable in a predefined sequence relative to the other filters as the second shaft is rotated in a forward direction. Each cam has a cam profile to ensure one of the filters remains in the optical light path until the next filter in the predefined sequence is driven into the optical light path. Each cam has a pin extending into a corresponding slot on the second shaft. The pin and corresponding slot are configured to allow the second shaft to be rotated in a reverse direction for a predetermined radial distance to allow a last remaining filter to be removed from the optical light path. The predetermined radial distance allows one of the filters to be rotated away from the optical light path without another filter being driven into the optical light path by rotation of the second shaft in a reverse direction.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a diagrammatic representation of an ophthalmic illumination system in accordance with one implementation of the disclosure;

FIG. 2 is a frontal view of an ophthalmic illumination filter selection mechanism in accordance with one implementation of the disclosure;

FIG. 3 is a cross-sectional view of a shaft and a cam mounted on the shaft in accordance with one implementation of the disclosure;

FIGS. 4 and 5 are frontal views of an ophthalmic illumination filter selection mechanism in accordance with one implementation of the disclosure; and

FIG. 6 is a side perspective view of an ophthalmic illumination filter selection mechanism in accordance with one implementation of the disclosure.

Corresponding reference numerals indicate corresponding parts throughout the several views of the drawings.

### DETAILED DESCRIPTION

Example embodiments will now be described more fully with reference to the accompanying drawings.

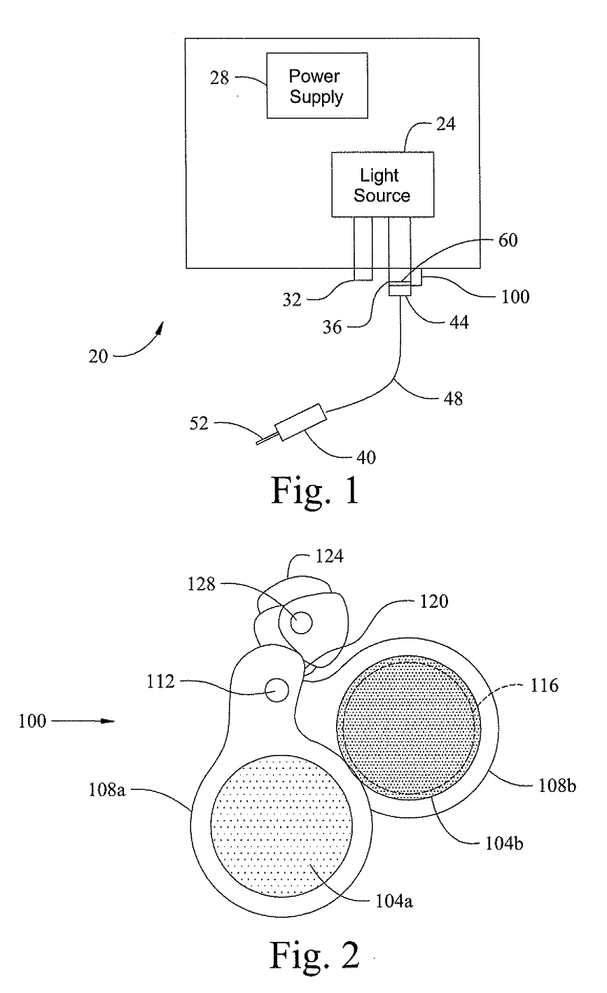

A diagrammatic representation of an ophthalmic illumination system is indicated generally in FIG. 1 by reference number 20. A light source 24 powered by a power supply 28 is capable of providing illumination via independently operable ports 32 and 36. A light probe 40 is connected to the port 36 at a proximal end 44 of a length 48 of optical fiber. Light from the light source 24 travels through the optical fiber 48 to and through a tip 52 of the probe 40. In accordance with one implementation of the disclosure, an ophthalmic illumination filter selection mechanism 100 may be selectively interposed by a user into a collimated section 60 of the light path between the light source 25 and the probe tip 52.

One configuration of the ophthalmic illumination filter selection mechanism 100 is shown in FIGS. 2, 4 and 5. The mechanism includes a plurality of, e.g., three, filters 104a-104c. Each filter is mounted in a corresponding holder 108a-108c. More than three filters may be provided, depending on the light filters desired. The holders 108a-108c are rotatably mounted on a shaft 112 that is substantially parallel to an optical light path 116 determined by the ophthalmic illumination system. The holders 108a-108c are rotatable on the shaft 112 into the optical light path 116. Each holder 108a-108c has a profile that includes a cam follower portion 120. A plurality of cams 124 are mounted on a shaft 128. Each cam 124 is configured to move a corresponding holder (108a, 108b or 108c) on the shaft 112 to position the corresponding filter (104a, 104b or 104c) in the optical light path 116. As the shaft 128 is rotated in a forward direction by a user, the filters 104a-104c are moved in a sequence determined by the arrangement of the cams 124 on the second shaft 128. In the present exemplary configuration, the cams 124 are radially spaced apart on the shaft 128 by intervals of about 120 degrees. If more or fewer filters are to be used the spaced intervals of the cams on the shaft in degrees of separation, should be 360/n, where n is the number of filters. A can profile of cams 124 is configured to ensure that one filter 104 is placed in the light path 116 before a previous filter 104 is removed from the light path 116. The cam profile of cams 124 therefore, prevent a transmission of unfiltered light to an eye. As shown in FIG. 2, the holder 108b is positioned such that its filter 104b is in the optical light path 116. The other two holders 108a and 108c, only one of which is visible in FIG. 2, are not in use and so are positioned outside the optical light path 116.

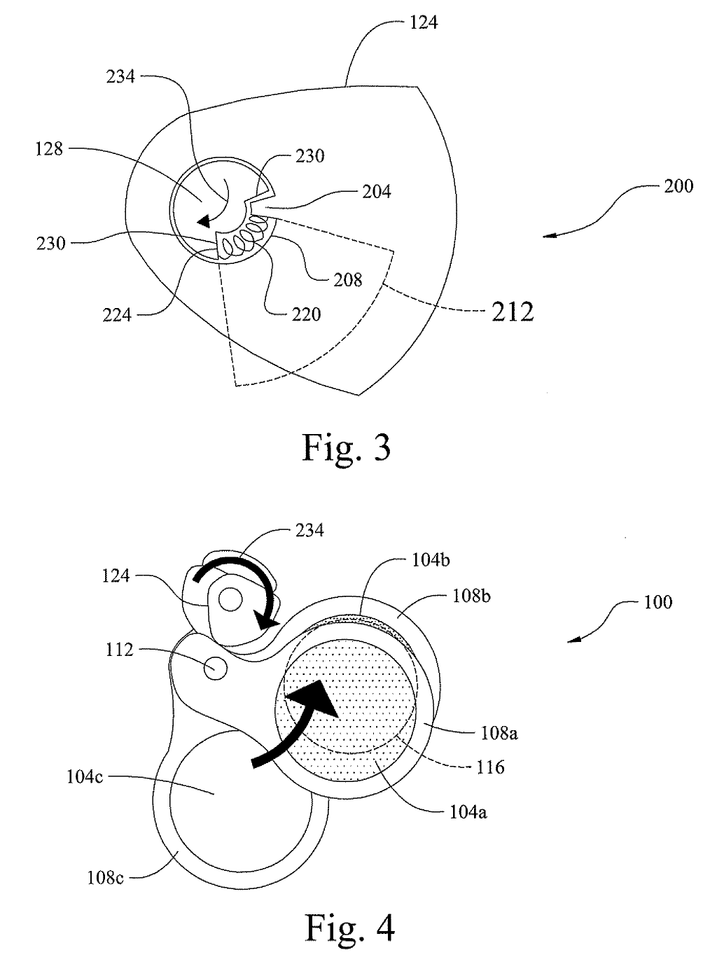

A cross-sectional view of the shaft 128 and a cam 124 mounted on the shaft 128 is indicated generally in FIG. 3 by reference number 200. Each cam 124 has a pin 204 extending into a corresponding slot 208 attached to the shaft 128. While FIG. 3 shows the slot 208 in shaft 128, if shaft 128 is sufficiently small in diameter it may be preferable to have driving spacers attached to shaft 128 with a corresponding slot formed in the driving spacer. The pin 204 and corresponding slot 208 are configured to provide a backlash in the cam 124. That is, the pin 204 and corresponding slot 208 are configured to allow the shaft 128 to be rotated in reverse (the direction of rotation for removing all filters from light path 116) for a predetermined radial distance 212 to allow a last remaining filter 104 to be removed from light path 116. In the present exemplary configuration, the backlash distance 212 is about seventy (70) degrees. This backlash allows shaft 128 to be rotated in reverse enough to remove a last one of the filters 104 from light path 116 so that unfiltered light is transmitted to the eye.

A spring 220 is provided between the pin 204 and an end 224 of the corresponding slot 208. The spring 220 holds the cam 124 at an end 230 of the slot 208, i.e., the slot end that corresponds to a forward direction 234 of rotation of the shaft 128. The spring 220 is configured to compress as the shaft 128 is rotated in a reverse direction 234 to remove a filter from the optical light path 116 and to unload as the shaft 128 is rotated in a forward direction.

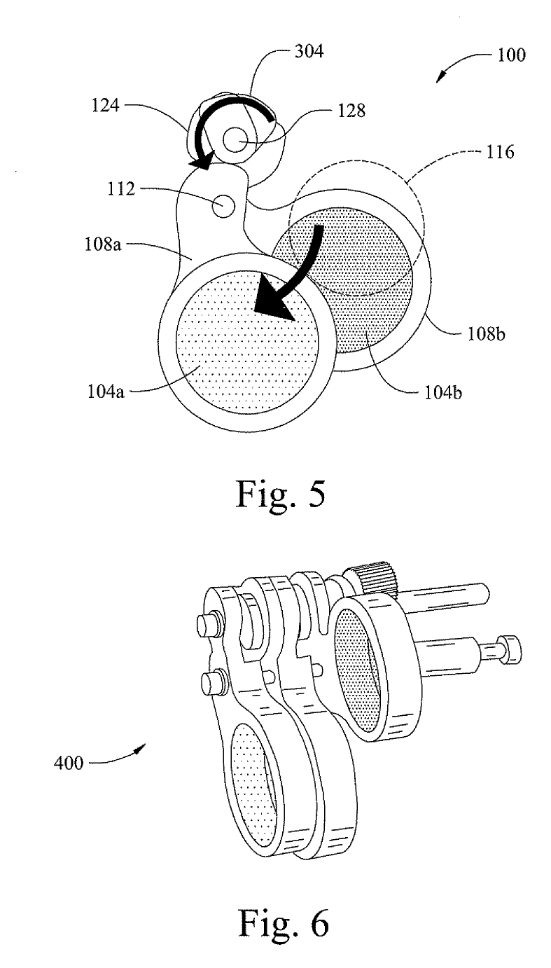

Operation of the filter selection mechanism 100 shall be described with reference to FIGS. 4 and 5. As shown in FIG. 4, the cam 124 corresponding to the holder 108a is being rotated in the forward direction 234 to rotate the holder 108a upward into the optical light path 116. As the cams 124 are rotated on the shaft 128, the cam profile of cams 124 allows the filter 104b to remain in the optical light path 116 until the filter 104a has been positioned in the light path. Thus, the filter 104a is engaged before the previous filter 104b is released. In such manner, essentially no unfiltered light is allowed to enter the light path 116 and is prevented from being transmitted to the eye, while the filters are being changed.

When it is desired to obtain unfiltered light output from the light source 24, the filter selection mechanism 100 is operated in reverse to release all filters, e.g., as shall be described with reference to FIG. 5. When, for example, the user rotates the shaft 128 in a reverse direction 304 to disengage the filter 104b currently in use, the cam 124 for the filter 104b is driven in the reverse direction 304, thereby causing the holder 108b to rotate downward out of the optical light path 116. Although the cam 124 for the previously engaged filter 104c comes into contact with the cam follower portion 120 of the corresponding holder 108c, the backlash provided in the cams 124 allows the shaft 128 to continue to rotate sufficiently for the filter 104c to move out of the light path 116.

A side perspective view of one configuration of an ophthalmic illumination filter selection mechanism is indicated generally in FIG. 6 by reference number 400. In various configurations, the foregoing mechanism can provide a surgeon with a selection of color filters as well as the option to use unfiltered light, in a space envelope much smaller than those required by conventional filter selection systems.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath”, “below”, “lower”, “above”, “upper” and the like, may be used herein for ease of description to describe one element or feature's relationship to another element(s) or feature(s) as illustrated in the figures. Spatially relative terms may be intended to encompass different orientations of a device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The foregoing description of the embodiments has been provided for purposes of illustration and description. It is not intended to be exhaustive or to limit the invention. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the invention, and all such modifications are intended to be included within the scope of the invention.

What is claimed is:

1. An ophthalmic illumination filter selection mechanism for use with an ophthalmic illumination system, the mechanism comprising:
a plurality of filters each mounted in a corresponding holder, each holder mounted on a first shaft and rotatable on the first shaft into an optical light path determined by the ophthalmic illumination system;
a plurality of cams mounted on a second shaft, each cam configured to move a corresponding holder on the first shaft to position the corresponding filter in the optical light path in a predefined sequence relative to the other filters as the second shaft is rotated in a forward direction;
each cam having a pin extending into a corresponding slot attached to the second shaft, the pin and corresponding slot configured to allow the second shaft to be rotated in a reverse direction for a predetermined radial distance to allow a last remaining filter to be removed from the optical light path; and
each cam having a cam profile to ensure one of the filters remains in the optical light path until a next filter in the predefined sequence is driven into the optical light path, thereby preventing unfiltered light from being transmitted to an eye.

2. The mechanism of claim 1, comprising three filters, the cams spaced on the second shaft at 120-degree intervals, and wherein the predetermined radial distance is 70 degrees.

3. The mechanism of claim 1, each cam comprising a spring between the pin and an end of the corresponding slot attached to the second shaft, the spring configured to compress as the second shaft is rotated in a reverse direction to remove a filter from the optical light path and to unload as the second shaft is rotated in a forward direction.

4. The mechanism of claim 1, wherein the second shaft is rotatable in a reverse direction to move a filter out of the optical light path.

5. The mechanism of claim 4, wherein the pin of each cam and corresponding slot are configured to allow one of the holders to be rotated away from the optical light path without another of the holders being driven into the optical light path by rotation of the second shaft in the reverse direction.

6. The mechanism of claim 1, wherein a portion of a profile of each holder is configured to follow the cam corresponding to the holder, and the second shaft is allowed to rotate over the predetermined radial distance when one of the cams comes into contact with its corresponding cam follower profile portion.

7. An ophthalmic illumination system comprising the filter selection mechanism of claim 1.

8. An ophthalmic illumination filter selection mechanism for use with an ophthalmic illumination system, the mechanism comprising:
a plurality of filters each mounted in a corresponding holder, each holder mounted on a first shaft and rotatable on the first shaft into a collimated portion of an optical light path determined by the ophthalmic illumination system;
a plurality of cams mounted on a second shaft, each cam drivable by the second shaft to move a corresponding holder on the first shaft to position the corresponding filter in the optical light path in a predefined sequence relative to the other filters as the second shaft is rotated in a forward direction;
each cam having a backlash configured to allow the second shaft to be rotated in a reverse direction for a radial distance to allow a last remaining filter to be removed from the optical light path;
the radial distance predetermined to allow one of the filters to be rotated away from the optical light path without another filter being driven into the optical light path by rotation of the second shaft in the reverse direction.

9. The mechanism of claim 8, wherein each cam has a pin extending into a corresponding slot attached to the second shaft, the pin and corresponding slot configured to provide the backlash.

10. The mechanism of claim 8, wherein each cam is configured to become spring-loaded as the second shaft is rotated in the reverse direction.

11. The mechanism of claim 8, comprising three filters, the cams spaced on the second shaft at 120-degree intervals, and wherein the radial distance is 70 degrees.

12. An ophthalmic illumination system comprising the filter selection mechanism of claim 8.

13. An ophthalmic illumination system having a light source and a light probe that receives light from the light source via an optical light path, the system comprising:
a color filter selection mechanism having three filters, each filter mounted in a corresponding holder rotatable on a first shaft into a collimated portion of the optical light path;
three cams mounted on a second shaft of the filter selection mechanism, each cam corresponding to one of the holders and drivable by the second shaft to move the corresponding holder on the first shaft to position the corresponding filter in the optical light path, the positioning performable in a predefined sequence relative to the other filters as the second shaft is rotated in a forward direction;
each cam having a pin extending into a corresponding slot attached to the second shaft, the pin and corresponding slot configured to allow the second shaft to be rotated in a reverse direction for a predetermined radial distance to allow a last remaining filter to be removed from the optical light path;
the radial distance predetermined to allow one of the filters to be rotated away from the optical light path without another filter being driven into the optical light path by rotation of the second shaft in a reverse direction; and
each cam having a cam profile to ensure one of the filters remains in the optical light path until a next filter in the predefined sequence is driven into the optical light path, thereby preventing unfiltered light from being transmitted to an eye.

14. The illumination system of claim 13, wherein the second shaft of the filter selection mechanism is rotated in the reverse direction by a user to obtain unfiltered light through the filter selection mechanism.

15. The illumination system of claim 13, wherein the cams are spaced apart from one another on the second shaft by equal radial intervals.

16. The illumination system of claim 13, wherein the predetermined radial distance is 70 degrees.
